(19) United States
(12) Patent Application Publication
(10) Pub. No.: US 2026/0325712 A1
(43) Pub. Date: Oct. 22, 2026

(54) CROSS-LINKED MATERIAL COMPRISING POLYSACCHARIDE MOIETIES INTERCONNECTED BY ESTER BONDS

(71) Applicant: MERZ AESTHETICS GMBH, Frankfurt am Main (DE)

(72) Inventors: Cleiton KUNZLER, Frankfurt am Main (DE); Colin DRABE, Frankfurt (DE); Radovan VUKICEVIC, Frankfurt am Main (DE); Radia EL-BANNA, Bad Vilbel (DE)

(21) Appl. No.: 19/485,795

(22) PCT Filed: May 23, 2024
(86) PCT No.: PCT/EP2024/064195

§ 371 Date: Nov. 19, 2025

(30) Foreign Application Priority Data
May 25, 2023 (EP) 23175399.7

(51) Int. Cl. A61L 27/20; A61L 27/52; C08B 37/08; C08J 3/24; C08K 5/357

(52) U.S. Cl. CPC A61L 27/20; A61L 27/52; C08B 37/0072; C08J 3/24; C08K 5/357; A61L 2430/34

(57) ABSTRACT

The present invention relates to a method for preparing a cross-linked material, comprising reaction of at least some carboxylic acid residues of polysaccharide moieties with at least some hydroxy residues of polysaccharide moieties to form ester bonds cross-linking polysaccharide moieties covalently with each other, wherein the reaction is triggered by one or more triazine-based activating agents. Furthermore, the invention refers to a cross-linked material obtainable from such method, compositions comprising such, and uses thereof.

The present invention relates to a method for preparing a cross-linked material, comprising reaction of at least some carboxylic acid residues of polysaccharide moieties with at least some hydroxy residues of polysaccharide moieties to form ester bonds cross-linking polysaccharide moieties covalently with each other, wherein the reaction is triggered by one or more triazine-based activating agents. Furthermore, the invention refers to a cross-linked material obtainable from such method, compositions comprising such, and uses thereof.

Facial and body re-shaping is of increasing interest. For example, filling of wrinkles of face and/or body, rejuvenation of the skin, breast reconstruction or augmentation, or soft-tissue augmentation of other kind is regularly of interest. In order to avoid the need of surgical interventions, a number of soft-tissue fillers that can be injected subcutaneously or within the deeper layers of the skin have been developed or are under development.

Soft-tissue fillers are typically gels such as hydrogels. The practitioner using such soft-tissue filler, in particular dermal fillers, typically desires that such fillers do not provoke toxic or immunologic adverse effects when administered under the conditions of interest, show good biocompatibility, are injectable without burden, and base on natural materials. Concomitantly, fillers should remain in a spatially defined area and have a sufficient stability in biological systems such as when being injected.

Suitable materials used for soft-tissue filling in the art are polysaccharides such as, e.g., hyaluronic acid (HA). Many polysaccharides such as hyaluronic acids have good biological acceptability. However, one significant drawback is that biodegradation of such material based on polysaccharides such as hyaluronic acid is comparably rapid and the filler material is not suitable for long-term solutions. It has limited longevity in subjects administered therewith, often of less than a desired minimum range of several months. When such polysaccharide is degraded rapidly in the body, the viscosity decreases undesirably rapidly and the filling effect is not sufficiently long lasting. Furthermore, storability and shelf life of hyaluronic acid-based filers is often limited. Stored product comprising unmodified polysaccharides often tend to partly degrade. Then, viscosity decreases and such stored and thus, partly degraded product that is then administered has an even shorter longevity in an administered subject.

Thus, there is the need for provision of further filling materials that have suitable biological stability.

It has been considered to cross-link polysaccharides such as hyaluronic acid with synthetic xenobiotic cross-linking agents such as, for example, the epoxide-based linker butanediol diglycidyl ether (BDDE) as for instance described in US-A 2012/0190644, WO 2015/149941 or WO 2020/030629, or di- or multinucleophilic functional cross-linkers such as described in US-A 2020/0140626. Such material have several drawbacks. For instance, residuals of such non-reacted or half-reacted bivalent linkers can be harmful and limit usability of the materials. There are maximally administrable contents of such reactive linkers and, thus, for safety reasons also for filler materials prepared by using such. When administered to a subject in need thereof and degraded in said subject, xenobiotic and undegradable or poorly degradable metabolites can be generated. This is generally not desired, in particular not in cosmetic and pharmaceutic uses. Thus, there is a desire to avoid such xenobiotic linker moieties.

To overcome these drawbacks, cross-linking with proteins, peptides and amino acids has been considered. In this context, it has been considered to cross-link polysaccharides such as hyaluronic acid with cross-linking proteins. For instance, hyaluronic acid was cross-linked with elastin as described in WO 2011/119468. Furthermore, polysaccharides were also cross-linked with fibroin such as described in WO 2022/268871. The application CN-A 105713211 describes the linkage of hyaluronate with high contents of the amino acid lysine containing two amino groups. In the absence of amino acids, it is not clear whether or not cross-linking is achieved and, if so, at which extend and which chemical bonds.

Such methods have the drawback that the presence of proteins, peptides or amino acids is not always desirable. The process is rather complex as it contains several components and, depending on the protein used, amide bonds within the protein sequence may be cleaved. In some cases, the proteins and peptides may bear undesired immunogenic responses. Thus, it is desirable to cross-link polysaccharide such as hyaluronic acid without such interconnecting proteins, peptides and amino acids.

In this context, it was considered to form amide bonds by cross-linking polysaccharides that bear carboxylic acid residues as well as amino groups with each other. For this purpose, polysaccharides such as glycosaminoglycans are modified to contain free amine groups which are conjugated to free carboxylic groups such as described in WO 2019/002369. Such processes, however, need an additional step of providing non-natural intermediate products such as provision of amino groups. Further, such obtained conjugates, undesirably contain amide bonds. It would be desirable to directly conjugate natural polysaccharides via ester bonds. In the art, there is an apparent prejudice that the formation of ester bonds between polysaccharides requires harsh activating agents such as carbodiimide activating agents (comprising the structural motif —N═C═N—) or 2-chloro-methylpyridinium iodide. For example, in EP-A 0341745 teaches that esterification can be achieved by means of 2-chloro-methylpyridinium iodide. JP-A 2019019201 teaches the use of carbodiimide activating agents such as N,N′-dicyclohexylcarbodiimide (DCC), diisopropylcarbodiimide (DIC), and 1-ethyl-3-(3-dimethylaminopropyl) carbodiimid (EDC). These reactive agents are undesirably irritant or even toxic and may bear further disadvantages.

When, on the other hand, such activating agents are omitted, esterification does either nor occur or is extremely slowly and ineffective, even at harsh pH ranges as shown in JP-A 2003/252905.

In view of above, there is still an unmet need for an efficient method for preparing a cross-linked material that requires only low procedural efforts, that avoids xenobiotic linker moieties introduced into the hydrogel and minimizes the residuals of toxic reagents. It is further desired to obtain ester bonds between polysaccharide moieties. Particularly desirable is a method that provides injectable hydrogel (e.g., usable as super-volumizer) for facial and body re-shaping.

Surprisingly, it has been found that a cross-linked material having beneficial properties can be obtained from a method comprising the reaction of at least some carboxylic acid residues of polysaccharide moieties with at least some hydroxy residues of polysaccharide moieties to form ester bonds cross-linking polysaccharide moieties covalently with each other, wherein the reaction is triggered by one or more triazine-based activating agents.

A first aspect relates to a method for preparing a cross-linked material, said method comprising:
(i) contacting the following components with each other:
   (A) polysaccharide moieties comprising carboxylic acid residues or salts thereof and hydroxy residues,
   (B) one or more triazine-based activating agents that effect reaction of carboxylic acid residues with hydroxy residues thereby forming ester bonds, and
   (C) one or more solvents; and
(ii) allowing reaction of at least some of the carboxylic acid residues of the polysaccharide moieties with at least some of the hydroxy residues of the polysaccharide moieties to form ester bonds cross-linking polysaccharide moieties covalently with each other; and
(iii) optionally purifying the cross-linked material obtained from step (ii), wherein there are more ester bonds cross-linking polysaccharide moieties covalently with each other than amide bonds cross-linking polysaccharide moieties covalently with each other,
wherein step (ii) is conducted in the absence of any activating agent other than triazine-based activating agents, in particular wherein step (ii) is conducted in the absence of carbodiimide activating agents, succinimidyl-based activating agents, glycidyl-based activating agents, paranitrophenol esters, and 2-chloro-methylpyridinium iodide.

It has been found that such cross-linked material bears unexpectedly beneficial properties. Viscosity may be widely maintained. Likewise, also enzymatic degradability may be desirably diminished. It was surprisingly found that xenobiotic linker structures as commonly used in the prior art can be avoided. The cross-linked material of the present invention may essentially consist of polysaccharide moieties, which can also found in nature. The cross-linked material of the present invention may also be designated as “cross-linked polysaccharide”, “self-cross-linked material” or “self-cross-linked polysaccharide”. The cross-linked material obtainable from the method of the present invention may have a long-lasting stability.

In aqueous environment, the obtained cross-linked material may form a hydrogel.

The cross-linked material may also be used to mimic an extracellular matrix and may, thus, induce cell proliferation and/or cellular migration and/or may serve as a scaffold for cells. It may be well used as a filler (e.g., dermal filer) which may be populated by cells. The cross-linked material of the present invention may have good shear-thinning properties. Preferably, it may have thixotropic properties. Thus, it may be less viscous when stressed. It may be injected very well, while still being rather viscous in its target area (when e.g., administered in a subcutaneous area). Comparably low extrusion forces are required. Gels of high viscosity and low extrusion force are obtainable. It may optionally serve as a super-volumizer.

The process of the present invention can be conducted without undue burden and with comparably low efforts.

The claimed method is particularly beneficial compared to procedures described in the prior art because it required only two educts (raw materials) in addition to a solvent, i.e., polysaccharide moieties and one or more triazine-based activating agents. Further components such as linkers are not required.

The omittance of xenobiotic linker structures such as, e.g., BDDE, may enable higher amounts of material administered to a subject, e.g., injected subdermally Furthermore, it may have longevity in a subject's body after administration.

The obtainable cross-linked material of the present invention may have a long shelf life and storability due to the avoidance of reactive groups. It is also thermally comparably stable.

As used herein, ester bonds may be understood in the broadest sense as generally understood in the art. Typically, an ester bond has the structure —O—CO— or —CO—O—, including tautomeric structures thereof.

As indicated above, the polysaccharide moieties are preferably predominantly linked with each other via ester bonds. In other words, more than 50% by mole, more than 75% by mole, or even more than 80% by mole, based on all bonds between polysaccharide moieties (interconnecting groups between polysaccharide moieties) may preferably be ester bonds.

In a preferred embodiment, more ester bonds than amide bonds are formed in the cross-linked material. In a preferred embodiment, the cross-linked material comprises a molar ratio of ester groups:amide groups of at least 25:1, preferably at least 50:1, in particular at least 100:1. In a preferred embodiment, the cross-linked material bears (essentially) no amine groups.

As used herein, the terms “residue” and “group” may be understood interchangeably.

As used herein, the terms “carboxylic acid residue” and “carboxy residue” may be understood interchangeably as generally understood in the art, i.e., as residue-COOH or the salt-COO-thereof.

In the polysaccharide moieties used as educts of the method of the present invention, there are preferably more hydroxy residues (—OH) than primary amine groups (—NH₂, including salts thereof, i.e., —NH₃⁺ (and counterions)).

In a preferred embodiment, the polysaccharide moieties comprise a molar ratio of hydroxy residues:primary amine groups of at least 25:1, preferably at least 50:1, in particular at least 100:1. In a preferred embodiment, the polysaccharide moieties bears (essentially) no amine groups.

As used in the context of the present invention, the term “polysaccharide moiety” may be understood the broadest sense as any moiety of a polysaccharide known in the art which comprises carboxylic acid residues or salts thereof and hydroxy residues. As used in the context of the present invention, the term “polysaccharide” may be understood the broadest sense as any polysaccharide in the art. According to the present invention, at least one polysaccharide moiety comprises at least one carboxylic acid residue or salt thereof. The polysaccharide moieties further comprises hydroxy residues. A polysaccharide may a naturally occurring polysaccharide that may be modified or may be a synthetic polysaccharide. In this context, a polysaccharide may be branched or unbranched. It will be understood that the term “polysaccharide moiety” may also include salts and modified forms thereof. In a preferred embodiment, the polysaccharide has not been oxidized.

Preferably, polysaccharide moieties are polymeric moieties of a weight average molecular weight (Mw) of at least 1 kDa (1000 Da), more preferably at least 5 kDa, even more preferably at least 10 kDa. In a preferred embodiment, the polysaccharide moieties have a weight average molecular weight of at least 50 kDa, preferably at least 200 kDa, in particular in the range of 500 to 4000 kDa. In a preferred embodiment, the polysaccharide moieties have a weight average molecular weight (Mw) in the range of from 10 to 10000 kDa, more preferably in the range of from 25 to 7500 kDa, in particular in the range of 50 to 4000 kDa.

The polysaccharide moieties may have any intrinsic viscosity (e.g. determined by a rheometer such as, e.g., described in the experimental section below). In a preferred embodiment, the polysaccharide moieties may have an intrinsic viscosity of 1 to 4 m³/kg (20° C., 1013 hPa, water). In a preferred embodiment, the polysaccharide moieties may have an intrinsic viscosity of 1.0 to 3.3 m³/kg (20° C., 1013 hPa, water). In a preferred embodiment, the polysaccharide moieties may have an intrinsic viscosity of 1.1 to 3.2 m³/kg, of 2.0 to 3.1 m³/kg, or of 2.5 to 3.0 m³/kg (each at 20° C., 1013 hPa, water).

In a preferred embodiment, the polysaccharide moieties have a weight average molecular weight of 1500 to 3500 kDa (1.5 and 3.5 MDa). More preferably, it may have a weight average molecular weight in the range of from 100 and 5000 kDa, of from 200 to 2000 kDa, of from 250 to 1500 kDa, of from 300 to 1000 kDa, of from 400 to 900 kDa or of from 500 to 900 kDa.

In a preferred embodiment, the polysaccharide moieties comprise one or more types of sugar acid moieties or salts thereof.

In a preferred embodiment, the polysaccharide moieties comprise one or more types of sugar acid moieties or salts thereof, wherein the one or more types of sugar acid moieties are selected from the group consisting of:
(B1) one or more uronic acid moieties, in particular selected from the group consisting of glucuronic acid moiety, galacturonic acid moiety, iduronic acid moiety, and combinations of two or more thereof;
(B2) one or more aldonic acid moieties, in particular selected from the group consisting of glyceric acid moiety, xylonic acid moiety, gluconic acid moiety, ascorbic acid moiety, and combinations of two or more thereof;
(B3) one or more ulosonic acid moieties, in particular selected from the group consisting of neuraminic acid moiety, ketodeoxyoctulosonic acid moiety, and combinations thereof; and/or
(B4) one or more aldaric acid moieties, in particular selected from the group consisting of tartaric acid moiety, meso-galactaric acid moiety, glucaric acid moiety, and combinations of two or more thereof.

In a preferred embodiment, the polysaccharide moieties comprise uronic acid moieties. In a preferred embodiment, the polysaccharide moieties comprise glucuronic acid moieties. In a preferred embodiment, the polysaccharide moieties comprise D-glucuronic acid moieties.

In a preferred embodiment, the polysaccharide moieties comprise or consist of D-sugar moieties. In an alternative embodiment, the polysaccharide moieties comprise or consist of L-sugar moieties. In an alternative embodiment, the polysaccharide moieties comprise or consist of a combination of D-sugar moieties and L-sugar moieties. For instance, in such combination, racemic mixture of sugar moieties may be comprised or specific sugar moieties are D-sugar moieties and others are L-sugar moieties. In a preferred embodiment, the polysaccharide moieties comprise or consist of one or more glycosaminoglycan moieties.

In a preferred embodiment, the polysaccharide moieties are selected from the group consisting of hyaluronic acid (HA) moieties, heparosan moieties, heparin, chondroitin sulphate, and mixtures of two or more thereof. In a preferred embodiment, the polysaccharide moieties comprise or consist of hyaluronic acid, glycerol-grafted hyaluronic acid, heparosan, chondroitin sulfate, and carboxymethyl cellulose. Such polysaccharides comprising carboxylic acid groups are also commercially available.

In a preferred embodiment, the polysaccharide moieties or consist of hyaluronic acid, glycerol-grafted hyaluronic acid, heparosan, chondroitin sulfate, and carboxymethyl cellulose. It will be understood that these may also comprise salts thereof.

In a preferred embodiment, the polysaccharide moieties comprise or consist of one or more hyaluronic acid moieties. It will be understood that this may also comprises salts thereof.

In a preferred embodiment, the cross-linked material of the present invention is a gel. In a preferred embodiment, the cross-linked material of the present invention is a polysaccharide-based gel. In a preferred embodiment, the cross-linked material of the present invention is a hyaluronic acid-based gel (HA gel). Such material may optionally also be designated as self-cross-linked hyaluronic acid (HA)

Hyaluronic acid (also: HA, hyaluronate, or hyaluronan) may be understood in the broadest sense as any hyaluronic acid in the art. It may be polysaccharide moiety that contains hyaluronic acid moieties (also hyaluronic acid units), preferably comprises at least 50 mol % of hyaluronic acid moieties, more preferably at least 75 mol %, even more preferably at least 80 mol %, even more preferably at least 90 mol %, referred to the whole content of saccharide moieties in the polysaccharide, of hyaluronic acid moieties. Hyaluronic acid may optionally comprise one or more saccharide moieties other than hyaluronic acid.

In a preferred embodiment, hyaluronic acid is a naturally glycosaminoglycan composed of linked repeating units of N-acetyl-D-glucosamine and D-glucuronic acid ([alpha-1,4-D-glucuronic acid-beta-1,3-N-acetyl-D-glucosamine]ₙ). Accordingly, the repeating/monomeric unit of hyaluronic acid may be exemplarity the following or a salt thereof:

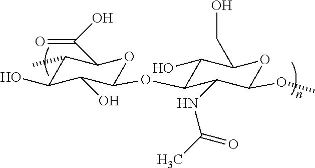

Accordingly, a repeating structure motif of hyaluronic acid may be exemplarity the following or a salt thereof:

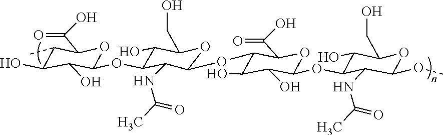

Hyaluronic acid may also embrace glycerol-grafted hyaluronic acid.

Hyaluronic acid may be used as described in WO 2022/268871. The weight average molecular weight (Mw) of hyaluronic acid in the context of the present invention is preferably at least 1 kDa (1000 Da), more preferably at least 5 kDa, even more preferably at least 10 kDa, even more preferably at least 50 kDa, even more preferably at least 100 kDa, even more preferably at least 200 kDa, even more preferably at least 300 kDa or more.

In a preferred embodiment, the polysaccharide moieties have a weight average molecular weight as laid out above.

Heparosan may be understood in the broadest sense as any heparosan. In a preferred embodiment, it may be such as described in WO 2015/149941. Heparosan (HEP) is a biopolymer belonging to the glycosaminoglycan (GAG) family of polysaccharides.

In humans, it is an intermediate product in the biosynthesis of heparin and heparin sulfate. The structure of heparosan is highly similar to that of hyaluronic acid (HA) since it has the same monosaccharide component sugars as hyaluronic acid and differs from HA only in that the beta-(1,3) glycosidic bond between the glucuronic acid (GlcUA) and the N-acetylglucosamine (GlcNAc) in HA is replaced by a beta-(1,4) glycosidic bond in HEP and in that the beta-(1,4) glycosidic bond between N-acetylglucosamine (GlcNAc) and the glucuronic acid (GlcUA) in HA is replaced by an alpha-(1,4) glycosidic bond in HEP:

GlcUA-beta-(1-4)-[GlcNAc-alpha-(1-4)-GlcUA-beta-(1-4)]ₙ-GlcNAc HEP

Typically, heparosan has excellent biocompatibility. Heparosan carries a high number of negative charges and hydroxy residues and is therefore highly hydrophilic, which increases tissue compatibility. Furthermore, due to the fact that heparosan polymers, even after modification, still comprise stretches that occur in natural heparan sulfate and heparin polymers, heparosan is typically non-immunogenic (e.g., does not induce antibodies). Moreover, due to the structural similarity between heparosan and hyaluronic acid, the same chemical modifications, including oxidation to aldehydes as that known for hyaluronic acid may be made on the functional groups. The molecular weight (Mw) of the heparosan polymers used in the context of the present invention may have any molecular weight.

Chondroitin sulfate may be understood in the broadest sense as generally understood in the art. In a n aqueous environment, it may be a structure comprising the following repeating/monomeric units (counterions are not depicted and may be any cations such as, e.g, sodium):

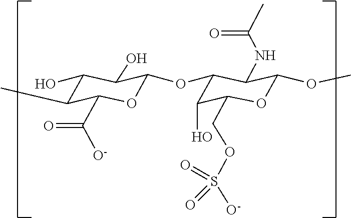

The polysaccharide moieties may be one or more types of polysaccharide moieties. These may be different in molecular size and/or may be different in the composition of monomeric units and/or may be different in chemical structure. Optionally, different polysaccharide moieties may be different types of polysaccharide moieties.

In one embodiment, the polysaccharide moieties, in particular hyaluronic acid moieties, have at least two different molecular weights each comprising primary amino residues or salts thereof. In other words, the polysaccharide moieties may also be a mixture of polysaccharide moieties of different molecular weight. In a preferred embodiment, the polysaccharide moieties have at least two different molecular weights and at least one polysaccharide moiety has, preferably at least two polysaccharide moieties, in particular all polysaccharide moieties, each have a molecular weight in the range of 10 to 10000 kDa, in the range of 100 to 10000 kDa, or in the range of 100 to 5000 kDa. in the range of from 100 to 3500 kDa, in the range of from 200 to 2000 kDa, in the range of from 250 to 1500 kDa, in the range of from 300 to 1000 kDa, in the range of from 400 to 900 kDa, or in the range of from 500 to 900 kDa.

In a preferred embodiment, the polysaccharide moieties have at least two different molecular weights and at least one polysaccharide moiety has, preferably at least two polysaccharide moieties, in particular all polysaccharide moieties, each have a molecular weight in the range of 1500 to 3500 kDa.

In a preferred embodiment, the total amount of polysaccharide moieties does not comprise more than 20% by weight, does not comprise more than 10% by weight, does not comprise more than 5% by weight, does not comprise more than 1% by weight, does not comprise more than 0.5% by weight, or does not comprise more than 0.1% by weight, based on the total amount of polysaccharide moieties, of polysaccharide moieties of molecular weights of less than 200 kDa.

In a preferred embodiment, the polysaccharide moieties comprise or consist of at least two hyaluronic acid moieties having at least two different molecular weights and at least one hyaluronic acid moiety has, preferably at least two hyaluronic acid moieties both have, in particular all hyaluronic acid moieties each have, a molecular weight in the range of 10 to 10000 kDa, in the range of 100 to 10000 kDa, or in the range of 100 to 5000 kDa. in the range of from 100 to 3500 kDa, in the range of from 200 to 2000 kDa, in the range of from 250 to 1500 kDa, in the range of from 300 to 1000 kDa, in the range of from 400 to 900 kDa, or in the range of from 500 to 900 kDa. In a preferred embodiment, the polysaccharide moieties comprise or consist of at least two hyaluronic acid moieties having at least two different molecular weights and at least one hyaluronic acid moiety has, preferably at least two hyaluronic acid moieties both have, in particular all hyaluronic acid moieties each have, a molecular weight in the range of 1500 to 3500 kDa.

The triazine-based activating agent may be used in any content suitable for cross-linking carboxylic acid residues of the polysaccharide moieties with at least some of the hydroxy residues of the polysaccharide moieties to form ester bonds thereby cross-linking polysaccharide moieties covalently with each other.

Typically, a triazine-based activating agent activates the carboxylic acid residues of the polysaccharide moieties. Thus, the content of triazine-based activating agent may be reasonably defined as molar equivalents (eq.) related to the carboxylic acid residues of the polysaccharide moieties. It will be understood that, in this context, carboxylic acid residues also embrace salts thereof. In other words, molar equivalents (eq.) of the triazine-based activating agent is defined as molar ratio of [triazine-based activating agent]:[carboxylic acid residues of the polysaccharide moieties]. For this purpose, in the context of sodium salt of hyaluronic acid (HA), in an approximation, it may be assumed that each HA monomeric/repeating unit when used as sodium salt (having a molecular weight (MW) of approximately 402 g/mol) bears one carboxylic acid residue. The crosslinking degree can be adjusted with different molar equivalents (eq.) of triazine-based activating agent.

In a preferred embodiment, the molar ratio of [triazine-based activating agent]:[carboxylic acid residues of the polysaccharide moieties] is in the range of 1:100 to 100:1, preferably in the range of 1:10 to 10:1, more preferably in the range of 1:5 to 5:1 or in the range of 1:2 to 2:1.

As used in the context of the present invention, a triazine-based activating agent may be any compound based on a triazine core structure that effects reaction of carboxylic acid residues with hydroxy residues thereby forming ester bonds. It will be understood that it is mainly meant that an activating agent is a compound that effects reaction of carboxylic acid residues of the polysaccharide moieties with hydroxy residues of the polysaccharide moieties thereby forming ester bonds.

In a preferred embodiment, an activating agent is typically not covalently included in the cross-linked material. Thus, it may be typically optionally removed from the cross-linked material of the present invention by any means such as, e.g., washing, filtration, etc.

A triazine-based activating agent may have any structure that comprises a triazine core that is suitable for cross-linking carboxylic acid residues of the polysaccharide moieties with at least some of the hydroxy residues of the polysaccharide moieties to form ester bonds thereby cross-linking polysaccharide moieties covalently with each other. For instance, a triazine-based activating agent may have the structure:

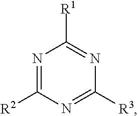

wherein R¹, R² and R³ are independently from each other any residue, such as, e.g., each independently a residue selected from the group consisting of hydrogen, deuterium, C₆-C₁₀-aryl, C₂-C₁₀-heteroaryl, linear or branched C₁-C₁₀-(cyclo)alkyl, linear or branched C₁-C₁₀-(cyclo)alkoxy, linear or branched C₁-C₁₀-hetero(cyclo)alkyl, linear or branched C₂-C₁₀-(cyclo)alkenyl, C₂-C₁₀-hetero(cyclo)alkenyl, linear or branched C₂-C₁₀-(cyclo)alkinyl, and C₂-C₁₀-hetero(cyclo)alkenyl, wherein each of the aforementioned residues is optionally substituted with one or more residues selected from the group consisting of halogen, C₆-C₁₀-aryl, C₂-C₁₀-heteroaryl, linear or branched C₁-C₁₀-(cyclo)alkyl, linear or branched C₁-C₁₀-hetero(cyclo)alkyl, linear or branched C₁-C₁₀-(cyclo)alkoxy, linear or branched C₂-C₁₀-(cyclo)alkenyl, C₂-C₁₀-hetero(cyclo)alkenyl, linear or branched C₂-C₁₀-(cyclo)alkinyl, and C₂-C₁₀-hetero(cyclo)alkenyl, or a salt thereof.

In a preferred embodiment, at least one or the residues R¹, R² and R³ is an optionally substituted morpholinium residue. In a preferred embodiment, at least one or the residues R¹, R² and R³ is a C₁-C₄-alkyl-morpholinium residue. In a preferred embodiment, at least one or the residues R¹, R² and R³ is a methylmorpholinium residue. In a preferred embodiment, at least one or the residues R¹, R² and R³ is an optionally substituted linear or branched C₁-C₁₀-(cyclo)alkoxy residue. In a preferred embodiment, at least one or the residues R¹, R² and R³ is an optionally substituted linear or branched C₁-C₄-alkoxy residue. In a preferred embodiment, at least one or the residues R¹, R² and R³ is an optionally substituted linear or branched methoxy residue.

In a preferred embodiment, the triazine-based activating agent has a molecular weight of not more than 1500 g/mol, of not more than 1000 g/mol, of not more than 500 g/mol, of not more than 400 g/mol,

In a preferred embodiment, the one or more activating agents are selected from the group consisting of 4-(4,6-dimethoxy-1,3,5-triazin-2-yl)-4-methylmorpholinium (DMTMM) or a salt thereof, and/or 2-chloro-4,6,-dimethoxy-1,3,5-triazine or a salt thereof, and combinations thereof.

In a preferred embodiment, the activating agent is 4-(4,6-dimethoxy-1,3,5-triazin-2-yl)-4-methylmorpholinium (DMTMM) or a salt thereof,

A salt of DMTMM is preferably a salt wherein the counter-ion is an anion that is cosmetically and/or pharmaceutically acceptable such as, e.g., chloride, acetate, bicarbonate (hydrogen carbonate), or a mixture of two or more anions.

In a preferred embodiment, the activating agent is 4-(4,6-dimethoxy-1,3,5-triazin-2-yl)-4-methylmorpholinium chloride (CAS No. 3945-69-5).

DMTMM is considered as having a comparably low and essentially negligible toxicity, is not cancerogenic, not mutagenic, and not teratogenic/reprotoxic in the generally used amounts. Thus, it is particularly well usable for preparing a soft-tissue filler such as a dermal or connective tissue filler.

When using DMTMM or a salt thereof as activating agent, 4-methylmorpholine (NMM) and/or 4,6-dimethoxy-1,3-5-triazine-2-ol (DMT) may be formed as degradation product(s). For example, the chloride salt of DMTMM may be used as as activating agent:

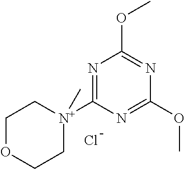 [SMILES: COc1nc(OC)nc([N+]2(C)CCOCC2)n1 ; Cl]

In a preferred embodiment, the method is further characterized in that it does not comprise at least one of the following:
(a) the use of interconnecting linker moieties inserted between the carboxylic acid residues or salts thereof and hydroxy residues of the polysaccharide moieties, in particular no peptidic or xenobiotic linker moieties;
(b) the use of a carbodiimide-based activating agent, in particular selected from the group consisting of N,N′-dicyclohexylcarbodiimide (DCC), diisopropylcarbodiimide (DIC), 1-ethyl-3-(3-dimethylaminopropyl) carbodiimid (EDC), and combinations of two or more thereof;
(c) the use of reactive groups selected from the group consisting of glycidyl ethers, maleimides, acid anhydrides, alkoxides and combinations of two or more thereof;
(d) polysaccharide moieties having primary amine groups, thiol groups, imide groups, imine groups, or epoxy groups.

In a preferred embodiment, the method is further characterized in that it does not comprise at least two, more preferably at least three, in particular all of the following:
(a) the use of interconnecting linker moieties inserted between the carboxylic acid residues or salts thereof and hydroxy residues of the polysaccharide moieties, in particular no peptidic or xenobiotic linker moieties;
(b) the use of a carbodiimide-based activating agent, in particular selected from the group consisting of N,N′-dicyclohexylcarbodiimide (DCC), diisopropylcarbodiimide (DIC), 1-ethyl-3-(3-dimethylaminopropyl) carbodiimid (EDC), and combinations of two or more thereof;
(c) the use of reactive groups selected from the group consisting of glycidyl ethers, maleimides, acid anhydrides, alkoxides and combinations of two or more thereof;
(d) polysaccharide moieties having primary amine groups, thiol groups, imide groups, imine groups, or epoxy groups.

The solvent may be any solvent that is suitable for reacting carboxylic acid residues or salts thereof and hydroxy residues of the polysaccharide moieties. In a preferred embodiment, the solvent is suitable for dissolving or suspending the polysaccharide moieties and dissolving the triazine-based activating agents. In a preferred embodiment, the solvent is suitable for dissolving the polysaccharide and the triazine-based activating agents. In a preferred embodiment, the solvent is a polar solvent. In a preferred embodiment, the solvent is a protic solvent. In a preferred embodiment, the solvent is a protic polar solvent.

In a preferred embodiment, the solvent comprises more than 50 wt. %, of at least 60 wt. %, of at least 70 wt. %, of at least 80 wt. %, of at least 90 wt. %, of at least 95 wt. %, or even 100 wt. %, referred to the total mass of the solvent, of one or more components selected from the group consisting of:
water;
one or more alcohols, preferably one or more C₁-C₅-alcohols, more preferably, one or more C₁-C₅-alcohols selected from the group consisting of methanol, ethanol, n-propanol, isopropanol, n-butanol (1-butanol), sec-butanol (2-butanol) isobutanol, (2-methylpropan-1-ol), tert-butanol (2-methylpropanol), pentan-1-ol, 2-methylbutan-1-ol, 3-methylbutan-1-ol, 2,2-dimethylpropan-1-ol, pentan-2-ol, 3-methylbutan-2-ol, pentan-3-ol, and/or 2-methylbutan-2-ol, and a combination of two or more thereof, in particular methanol and/or ethanol;
one or more primary amines, in particular in particular one or more C₁-C₅-amines;
one or more carbonic acids, preferably one or more C₁-C₅-carbonic acids selected from the group consisting of formic acid, acetic acid, propionic acid, butyric acid, valerianic acid, isovalerianic acid, in particular formic acid and/or acetic acid; one or more primary or secondary amides, preferably one or more C₁-C₅-amides, in particular formamide;
one or more sulfoxides, preferably one or more C₁-C₅-amides, in particular dimethyl sulfoxide (DMSO);
and a combination of two or more thereof.

In a preferred embodiment, the solvent is an aqueous solution. An aqueous solvent may be understood in the broadest sense as a solvent that comprises a water content by weight of more than 50 wt. %, of at least 60 wt. %, of at least 70 wt. %, of at least 80 wt. %, of at least 90 wt. %, of at least 95 wt. %, or even 100 wt. %, referred to the total mass of the solvent. In one embodiment of the present invention, an aqueous buffer comprises, in addition to water, one or more components selected from the group consisting of one or more alcohols (in particular one or more C₁-C₅-alcohols such as, e.g., methanol, ethanol, n-propanol, isopropanol, n-butanol (1-butanol), sec-butanol (2-butanol) isobutanol, (2-methyl-propan-1-ol), tert-butanol (2-methylpropanol), pentan-1-ol, 2-methylbutan-1-ol, 3-methylbutan-1-ol, 2,2-dimethylpropan-1-ol, pentan-2-ol, 3-methylbutan-2-ol, pentan-3-ol, and/or 2-methylbutan-2-ol), one or more primary amines (in particular one or more C₁-C₅-amines), one or more carbonic acids (in particular one or more C₁-C₅-carbonic acids such as, e.g., formic acid, acetic acid, propionic acid, butyric acid, valerianic acid, isovalerianic acid), one or more primary or secondary amides (in particular one or more C₁-C₅-amides such as, e.g., formamide), one or more sulfoxides (in particular one or more C₁-C₅-amides such as, e.g., dimethyl sulfoxide (DMSO)), one or more inorganic or organic cations (in particular one or more inorganic or organic cations of a molecular weight or less than 1000 Da, in particular alkali cations or earth alkali cations, other metal cations, protons, ammonium cations, etc.), one or more inorganic or organic anions (in particular one or more inorganic or organic anions of a molecular weight or less than 1000 Da, in particular chlorine, sulfate, etc.), one or more silicates, and a combination of two or more thereof.

In a preferred embodiment, the solvent is an aqueous buffer (e.g., phosphate buffered saline (PBS), Tris buffer, borate buffer, acetic acid-buffered buffer, etc.). In a preferred embodiment, a hydroalcoholic solvent is used such as, e.g., a mixture of water with ethanol, methanol, propanol, butanol, and/or pentanol. In a preferred embodiment, water is used as solvent. In a preferred embodiment, the solvent is an aqueous buffer having a pH in the range of 5 to 9, in particular 6 to 8. For example, the solvent may be an aqueous buffer having a pH in the range of 6.1 to 7.9, of 6.5 to 7.7, of 6.7 to 7.5, or of 7.0 to 7.5,

It was surprisingly found that the reaction steps, including pre-swelling, does not need alkalization and the reaction takes place at essentially neutral pH. This rebutted the prejudice in the art indicating that either strongly acidic or basic pH ranges were required.

Water as used herein, may be understood in the broadest sense. Preferably, water is deionized water, distilled water, or tap water, in particular deionized water or distilled water.

In a preferred embodiment, the method does not comprise a purification step (iii). In another embodiment, the method comprises at least one purification step (iii). In one embodiment, the method comprises at least one step (iii) of purifying the cross-linked material by filtration, washing and/or dialysis, in particular crossflow filtration, diafiltration and/or dead-end filtration;

Dialysis may be conducted by any means. In a preferred embodiment, dialysis is using dialysis membranes having a molecular weight cut-off (MWCO) of 1 to 25 kDa, of 5 to 20 kDa, or of 10 to 15 kDa. Purification time may be adapted to the molecule structures and may be in the range of at least 1 hour, at least 6 hours, 12 to 78 hours, 1 to 7 days, or 1 to 3 days. The swelling ratio may be optionally controlled by the weight of the membranes and the dialysis may be stopped once the product shows the desired cross-linked material concentration.

It will be understood that filtration may be crossflow filtration, dead-end filtration, or a combination of both. Filtration may be performed by any means. In the context of filtration, the a filter may have any pore size suitable for purifying the cross-linked material, i.e., preferably withholding the cross-linked material and allowing the passage of reactants and optionally of non-reacted polysaccharides. Optionally, the pore size may be in a range of 5 nm to 2 μm, more particularly a pore size of 30 nm to 600 nm, more particularly a pore size of 80 nm to 300 nm, particularly a pore size of 5 nm to 60 nm. A filter may be of any material such as, e.g., ceramic, metal, polymer material, or a combination thereof.

Optionally, filtration may be dynamic filtration such as, e.g., described in WO 2020/030629. Accordingly, step (iii) may optionally comprise dynamic filtration of the cross-linked material, optionally, comprising the following steps:
a) transferring the cross-linked material in a dynamic filtration device which is equipped with semipermeable filter disc(s) and diafiltrating the gel comprising the steps of:
   i) concentrating the cross-linked material by applying a rotational speed within the range of 20 1/min to 500 1/min and a overpressure within the range of 0.5 to 6 bar to a predetermined concentration; or pumping the cross-linked material directly into the process chamber of the dynamic filtration device;
   ii) conducting a diafiltration to reduce unwanted molecules by applying a rotational speed within the range of 20 1/min to 500 1/min and a overpressure within the range of 0.5 to 6 bar;
b) optionally adding a mixture comprising a non-cross-linked material and water to the cross-linked material.

In one embodiment, the dynamic filtration device is equipped with 1 to 10 semipermeable filter disc(s). In DCF, any rotational speed and pressure may be used, such as, e.g., a rotational speed within the range of 20 1/min to 500 1/min and a pressure within the range of 0.5 to 3 bar. In DCF, any concentration may be used such as e.g., 10 to 70 mg/g.

In an embodiment of the present invention, crossflow filtration (also: crossflow filtration) is dynamic crossflow filtration (DCF). Thus, in an embodiment, the method may be further characterized in that it comprises a step (iii) of purifying the cross-linked material by DCF. This is exemplified below. Optionally, DCF may be such as described in WO 2020/030629.

Optionally, one or more further components (e.g., one or more local anesthetics (e.g., as laid out below such as, e.g., lidocaine), one or more cell proliferation factors, one or more dyes, and combinations of two or more thereof) may be added before, during or after conducting step (iii) of purifying the cross-linked material by filtration, washing and/or dialysis.

In one embodiment, steps (i) and (ii) are conducted in a single batch. In another embodiment, steps (i) and (ii) are conducted in separate batches.

Steps (i) and (ii) and optional step (iii) any be each conducted at any temperature range. In a preferred embodiment, step (i) is conducted at a temperature in the range of from 5 to 90° C., preferably 18 to 60° C., in particular more preferably 18 to 25° C. or 20° C. to 50° C. In a preferred embodiment, step (ii) is conducted at a temperature in the range of from 5 to 90° C., preferably 18 to 60° C., in particular 20° C. to 50° C. In a preferred embodiment, as far as conducted step (iii) is conducted at a temperature in the range of from 5 to 90° C., or 18 to 60° C., or 20° C. to 50° C. In a preferred embodiment, steps (i) and (ii) and optional step (iii) are conducted at a temperature in the range of from 5 to 90° C., preferably 18 to 60° C., in particular 20° C. to 50° C. In a preferred embodiment, steps (i) and (ii) are conducted at temperatures that do not different in more than 10° C., do not different in more than 5° C., or do not different in more than 2° C. For instance, steps (i) and/or (ii) and/or step (iii) may be conducted at a temperature of (approximately) 18° C., of (approximately) 19° C., of (approximately) 20° C., of (approximately) 21° C., of (approximately) 22° C., of (approximately) 23° C., of (approximately) 24° C., of (approximately) 25° C., of (approximately) 26° C., of (approximately) 27° C., of (approximately) 28° C., of (approximately) 29° C., or of (approximately) 30° C. Steps (i) and (ii) and optional step (iii) may be conducted at any pressure. For example, pressure may be ambient pressure (e.g., often approximately 970 to 1100 hPa outer pressure).

Step (i) of contacting the components with each other may be conducted by any means. In a preferred embodiment, the method is further characterized in that step (i) involves the mixing of the components, i.e., the polysaccharide and the one or more activating agents, and one or more solvents, and optional one or more further components. Such mixing may be conducted by any means such as, e.g., by means of stirring and/or shaking.

In a preferred embodiment, the polysaccharide moieties are mixed with the solvents or parts of the solvent before contacting it with the one or more activating agents. This step may contain an incubation of the mixture of the polysaccharide moieties and the solvents or parts of the solvent (also: pre-swelling). For instance, such optional incubation step may be conducted (e.g., at a temperature of 4 to 40° C.) for at least 10 min, for 30 min to 7 days, for 1 hour to 2 days, for 6 to 36 hours, or for 12 to 24 hours. The one or more activating agents may be optionally premixed with parts of the solvent and subsequently added to the mixture of the polysaccharide moieties and the solvents or parts of the solvent.

The reaction step (ii) may be conducted as long as suitable for achieving the desired reaction of the carboxylic acid residues or salts thereof with the hydroxyl residues. Step (ii) may be conducted for any time suitable for this purpose. Optionally, step (ii) may be conducted for 1 min to 1 week or longer, 2 min to 5 days, 3 min to 4 days, 5 min to 72 hours, 5 min to 24 hours, 10 min to 12 hours, 30 min to 6 hours, 1 hour to 5 hours, or 2 to 4 hours. Step (ii) may be conducted for at any temperature suitable for this purpose such as, e.g., at 0° C. to 100° C., at 4° C. to 95° C., at 10° C. to 70° C., at 15° C. to 30° C., at 18 to 25° C., at 20° C. to 70° C., at 20° C. to 40° C., or at 60° C. to 70° C. In a preferred embodiment, the step (ii) is conducted for not more than 72 h, for not more than 48 h, or for not more than 24 h. In a preferred embodiment, the step (ii) is conducted for at least 10 min, preferably for 15 min to 48 h, in particular for 30 min to 18 h. In a preferred embodiment, the method is further characterized in that the step (ii) is conducted at a temperature in the range of from 5 to 90° C., preferably 18 to 60° C., in particular 20° C. to 50° C., for at least 10 min, preferably for 15 min to 48 h, in particular for 30 min to 18 h. In a preferred embodiment, the method is further characterized in that the step (ii) is conducted at a temperature in the range of from 5 to 90° C. for at least 10 min. In a preferred embodiment, the method is further characterized in that the step (ii) is conducted at a temperature in the range of 18 to 60° C. for 30 min to 18 h. Step (ii) may be conducted at any pH. For example, it may be conducted at a pH in the range of 5 to 9, in particular 6 to 8, of 6.1 to 7.9, of 6.5 to 7.7, of 6.7 to 7.5, or of 7.0 to 7.5,

During reaction step (ii), the polysaccharide moieties may be used at any concentration range. In a preferred embodiment, 0.01 to 800 mg/mL, or 0.1 to 500 mg/mL, or 0.5 to 200 mg/ml of polysaccharide moieties is used. In a preferred embodiment, 1 to 100 mg/mL, or 2 to 75 mg/mL, or 5 to 50 mg/mL, or 20-25 mg/mL, or 25 to 35 mg/mL, of polysaccharide moieties is used.

The optional purification step (iii), if conducted, may be conducted for any time suitable for this purpose. Optionally, step (iii) may be conducted for 1 min to 1 week or longer, 2 min to 5 days, 3 min to 4 days, 5 min to 72 hours, 5 min to 24 hours, 10 min to 12 hours, 30 min to 6 hours, 1 hour to 5 hours, or 2 to 4 hours. Step (iii) may be conducted for at any temperature suitable for this purpose such as, e.g., at 0° C. to 100° C., at 4° C. to 95° C., at 10° C. to 70° C., at 15° C. to 30° C., at 18 to 25° C., at 20° C. to 70° C., at 20° C. to 40° C., or at 60° C. to 70° C.

In a preferred embodiment, the method is further characterized in that:
(a) it comprises a step (iii) of purifying the cross-linked material by filtration, washing and/or dialysis, in particular crossflow filtration, diafiltration and/or dead-end filtration;
(b) steps (i) and (ii) are conducted in a single batch;
(c) steps (i) and (ii) and optional step (iii) are conducted at a temperature in the range of from 5 to 90° C., preferably 18 to 60° C., in particular 20° C. to 50° C.; and/or
(d) the step (ii) is conducted for at least 10 min, preferably for 15 min to 48 h, in particular for 30 min to 18 h.

In a preferred embodiment, the method comprises:
(i) contacting the following components with each other:
   (A) polysaccharide moieties comprising carboxylic acid residues or salts thereof and hydroxy residues having a weight average molecular weight of at least 50 kDa,
   (B) one or more triazine-based activating agents that effect reaction of carboxylic acid residues with hydroxy residues thereby forming ester bonds, in particular wherein the activating agent is 4-(4,6-dimethoxy-1,3,5-triazin-2-yl)-4-methylmorpholinium or a salt thereof; and
   (C) an aqueous solvent, preferably an aqueous solvent having a pH in the range of 5 to 9, in particular 6 to 8; and
(ii) allowing a reaction of at least some of the carboxylic acid residues with at least some of the hydroxy residues to form ester bonds cross-linking polysaccharide moieties covalently with each other; and
(iii) optionally purifying the cross-linked material obtained from step (ii).

In an optional further step, the obtained cross-linked material may be optionally treated further. For instance, it may be optionally homogenized and/or passed through a sieve (screening). In this context, the terms sieve, filter and mesh may be understood interchangeably. This may result in a particularly homogeneous material (e.g., hydrogel). In an optional further step, the cross-linked material may be subjected to sterilization.

In a preferred embodiment, the method comprises the following steps:
(i) contacting the following components with each other:
   (A) polysaccharide moieties comprising carboxylic acid residues or salts thereof and hydroxy residues,
   (B) one or more triazine-based activating agents that effect reaction of carboxylic acid residues with hydroxy residues thereby forming ester bonds, and
   (C) an aqueous solvent, preferably an aqueous solvent,
   wherein the polysaccharide moieties are pre-mixed with parts of the aqueous solvent and incubated (e.g., for 30 min to 7 days);
(ii) allowing a reaction of at least some of the carboxylic acid residues with at least some of the hydroxy residues to form ester bonds cross-linking polysaccharide moieties covalently with each other for 10 min to 2 days at temperature of 20 to 60° C.; and
(iii) optionally purifying the cross-linked material obtained from step (ii) by dialysis; and
(iv) optionally homogenizing and/or passing through a sieve and/or admixing an anesthetic (e.g., lidocaine) and/or optionally sterilization.

In a preferred embodiment, the method comprises:
(i) contacting the following components with each other:
   (A) polysaccharide moieties comprising carboxylic acid residues or salts thereof and hydroxy residues having a weight average molecular weight of at least 50 kDa,
   wherein the polysaccharide moieties are pre-mixed with parts of the aqueous solvent and incubated (e.g., for 30 min to 7 days),
   (B) one or more triazine-based activating agents that effect reaction of carboxylic acid residues with hydroxy residues thereby forming ester bonds, in particular wherein the activating agent is 4-(4,6-dimethoxy-1,3,5-triazin-2-yl)-4-methylmorpholinium or a salt thereof; and
   (C) an aqueous solvent, preferably an aqueous solvent having a pH in the range of 5 to 9, in particular 6 to 8; and
(ii) allowing a reaction of at least some of the carboxylic acid residues with at least some of the hydroxy residues to form ester bonds cross-linking polysaccharide moieties covalently with each other for 10 min to 2 days at temperature of 20 to 60° C.; and
(iii) optionally purifying the cross-linked material obtained from step (ii) by dialysis; and
(iv) optionally homogenizing and/or passing through a sieve and/or admixing an anesthetic (e.g., lidocaine) and/or optionally sterilization.

It will be understood that the cross-linked material obtainable from a method of the present invention bears special technical characteristics such as structural characteristics and the absence of harsh (bi) functional linkers and activating agents.

Accordingly, a further aspect of the present invention relates to a cross-linked material obtainable from a method of the present invention.

It will be understood that the definitions and preferred embodiments as laid out in the context of the method of the present invention mutatis mutandis apply to the cross-linked material of the present invention.

In an embodiment of the present invention, the cross-linked material has been prepared by using DMTMM as activating agent. In an embodiment of the present invention, the cross-linked material, in particular before final purification, comprises:
(a) 4-(4,6-dimethoxy-1,3,5-triazin-2-yl)-4-methylmorpholinium or a salt thereof,
(b) N-methylmorpholinium or salt thereof; and/or
(c) 4,6-dimethoxy-1,3,5-triazin-2-ol or a tautomer or salt thereof.

In an embodiment of the present invention, the cross-linked material, in particular before final purification, comprises NMM and/or DMT. In an embodiment of the present invention, the cross-linked material, in particular before final purification, comprises up to 0.1% by weight, preferably 0.01 to 1000 ppm, 0.1 to 100 ppm, or 1 to 50 ppm of NMM and/or DMT, referred to the total weight of the cross-linked material (gel).

A further aspect of the present invention relates to a cross-linked material comprising or consisting of one or more hyaluronic acid moieties having a weight average molecular weight of at least 50 kDa, wherein the hyaluronic acid moieties are covalently cross-linker with each other via ester bonds without an interconnecting linker structure,
further characterized in that the cross-linked material does not comprise diimide groups, epoxy groups or xenobiotic linker moieties.

As used herein, the term “without an interconnecting linker structure” may be understood in the broadest sense in that no further chemical moiety that does not originate from (also: is not present in) polysaccharide moieties (e.g., hyaluronic acid moieties) is introduced into the chemical structure that conjugates polysaccharide moieties with ester bonds. In other words, the ester bonds are preferably formed from inclusion of an oxygen atom originating from polysaccharide moieties (e.g., hyaluronic acid moieties) and from inclusion of a carbon atom originating from polysaccharide moieties (e.g., hyaluronic acid moieties).

In a preferred embodiment, the cross-linked material is further characterized in that it does not comprise imide groups or diimide groups. In a preferred embodiment, the cross-linked material is further characterized in that it does not comprise imine groups. In a preferred embodiment, the cross-linked material is further characterized in that it does not comprise epoxy groups. In a preferred embodiment, the cross-linked material is further characterized in that it does not comprise xenobiotic linker moieties groups.

In a preferred embodiment, the cross-linked material is further characterized in that it does not comprise:
(a) diimide groups
(b) imide groups;
(c) imine groups;
(d) epoxy groups; and/or
(e) xenobiotic linker moieties,
interconnecting polysaccharide moieties.

In a preferred embodiment, the cross-linked material is further characterized in that it does not comprise:
(a) diimide groups
(b) imide groups;
(c) imine groups;
(d) epoxy groups; and
(e) xenobiotic linker moieties,
interconnecting polysaccharide moieties.

In a preferred embodiment, the cross-linked material is obtained from a method of the present invention.

Depending on the intended use of the cross-linked material, it may be immediately used or may be stored. For instance, it may be stored at any condition suitable for this purpose such as, e.g., at ambient temperature (e.g., 18 to 30° C., preferably 18 to 25° C.), in a fridge (e.g., at 0 to 15° C., preferably 3 to 10° C.), in a freezer (e.g., −30 to 0° C., preferably −25 to −10° C.), in a deep freezer (e.g., −100 to −200° C., preferably −90 to −55° C.), on liquid nitrogen, on dry ice, or even one or more liquid noble gases. It may be stored at dry state, as a hydrogel, as a gel containing other non-aqueous solvents, and/or as a suspension, emulsion, colloid or solution.

The solvent used in steps (i) and (ii) and/or optionally in step (iii) may optionally be partly or completely removed. As the used components, including the activating agent, may bear a comparably low toxicity, the solvent used in steps (i) and (ii) and/or optionally in step (iii) may alternatively optionally also be maintained in the cross-linked material. If removed, the solvent used in steps (i) and (ii) and/or optionally in step (iii) may be removed by any means such as, e.g., by replacing it by another solvent (e.g., another aqueous buffer) and/or by evaporation.

The cross-linked material may be used for any purpose and in any form. For instance, it may be gel when comprising a liquid or viscous medium in its voids. In general, such liquid or viscous medium may be any liquid or viscous medium that does not disintegrate the cross-linked material. Preferably, the liquid or viscous medium is an aqueous liquid. As indicated above, the cross-linked material of the present invention can be used for preparing a hydrogel.

Accordingly, a further aspect of the present invention relates to a hydrogel comprising:
(A) a cross-linked material of the present invention; and
(B) an aqueous solution; and
(C) optionally one or more further pharmaceutically or cosmetically acceptable agents, in particular one or more local anesthetics.

It will be understood that the definitions and preferred embodiments as laid out in the context of the method and the cross-linked material of the present invention mutatis mutandis apply to the hydrogel of the present invention.

Accordingly, the present invention also relates to a method for preparing a hydrogel. Optionally, such method may comprise a step of adding an aqueous buffer to a purified cross-linked material.

The cross-linked material and/or the hydrogel of the present invention may be used for any purpose. For instance these may be used as an injectable composition.

Accordingly, a further aspect of the present invention relates to an injectable composition comprising a cross-linked material or a hydrogel of the present invention, wherein the cross-linked material preferably is a super-volumizer.

It will be understood that the definitions and preferred embodiments as laid out in the context of the method, the cross-linked material and the hydrogel of the present invention mutatis mutandis apply to the injectable composition of the present invention.

In a preferred embodiment, the cross-linked material, hydrogel and/or the injectable composition of the present invention is usable as a soft-tissue filler, in particular a dermal filler or a connective tissue filler.

A liquid or viscous carrier according the present invention as comprised in the injectable composition may be any injectable carrier. Typically, the liquid or viscous carrier is a pharmaceutically and/or cosmetically acceptable carrier, therefore, a carrier that is non-toxic to the mammal, in particular a human, when administered to the mammal in the sense of the present invention. The liquid or viscous carrier may preferably comprise or consist of one or more solvents such as, e.g., water, an aqueous buffer (e.g., a saline or phosphate buffered saline), dimethyl sulfoxide (DMSO), ethanol, vegetable oil, paraffin oil or combinations thereof. More preferably, the liquid or viscous carrier comprises or consists of an apyrogenic isotonic buffer, more particularly a physiological saline solution or a buffered physiological saline solution.

An optionally present further components may be any components. For example, such further component may be selected from the group consisting of one or more local anesthetics, one or more cell proliferation factors, one or more dyes, and combinations of two or more thereof.

Such further components may be added at any time such as before, during or after purifying the cross-linked material. For instance, one or more further components may be added during conducting a purifying step (iii). In another embodiment of the present invention, one or more further components may be added to the prepared and optionally purified cross-linked material.

A local anesthetic may make injection into an individual more comfortable. A cell proliferation factor may improve cellular invasion into an administered cross-linked material of the present invention. A dye may either improve localization of the injection (e.g., a pharmaceutically acceptable fluorescent dye like fluorescein or rhodamine) or may improve invisibility of the otherwise whitish cross-linked material (e.g., by rendering it flesh-colored). Any other pharmaceutically active compound may also be added. Then, the cross-linked material of the present invention may also serve as a retard form for administration.

Suitable local anesthetics for use herein include, but are not limited to, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and salts thereof. Combinations of two or more of the mentioned anesthetic agents, for example a combination of lidocaine and other “caine”-anesthetic(s) like prilocaine, may also be used herein.

Depending on the intended use of the cross-linked material, hydrogel and/or the injectable composition of the present invention, it can be provided in different packaging. It may be stored at any condition suitable for this purpose such as, e.g., at ambient temperature (e.g., 18 to 30° C., preferably 18 to 25° C.), in a fridge (e.g., at 0 to 15° C., preferably 3 to 10° C.), in a freezer (e.g., −30 to 0° C., preferably −25 to −10° C.), in a deep freezer (e.g., −100 to −200° C., preferably −90 to −55° C.), on liquid nitrogen, on dry ice, or even one or more liquid noble gases. For instance, it may be provided in a vial, in a syringe. It may be administered to a subject via injection (e.g., via a syringe or a drip). It may be stored at dry state, as a hydrogen, as a gel containing other non-aqueous solvents, and/or as a suspension, emulsion, colloid or solution.

The present invention also refers to the use of an injectable composition according to the present invention for cosmetic applications. More preferably, the present invention also refers to the use of an injectable composition according to the present invention for cosmetic applications comprising facial and body re-shaping and rejuvenation.

As used herein, cosmetic applications and cosmetic uses may be understood in the broadest sense as any non-therapeutic application intended for improving appearance. It may also be designated as aesthetic application and aesthetic use, respectively. It will be understood that, in the context of the present invention, a cosmetic application and cosmetic use may include any administration route that may optionally include an injection of the respective material and/or topical administration of the respective material or another suitable administration route.

A further aspect of the present invention relates to the use of an injectable composition of the present invention for cosmetic applications comprising facial and body re-shaping and rejuvenation, preferably including filling of wrinkles, improving facial lines, breast reconstruction or augmentation, rejuvenation of the skin, buttocks augmentation, remodeling of cheekbones, soft-tissue augmentation, filling facial wrinkles, improving glabellar lines, improving nasolabial folds, improving marionette lines, improving buccal commissures, improving peri-lip wrinkles, improving crow's feet, improving subdermal support of the brows, malar and buccal fat pads, improving tear troughs, improving nose appearance, augmentation of lips, augmentation of cheeks, augmentation of peroral region, augmentation of infraorbital region, resolving facial asymmetries, improving jawlines, augmentation of chin, or a combination of two or more thereof.

It will be understood that the definitions and preferred embodiments as laid out in the context of the method, the cross-linked material, hydrogel and the injectable composition of the present invention mutatis mutandis apply to the use of the present invention.

This use may be a therapeutic and/or cosmetic use. In preferred embodiment, the present invention relates to the use of an injectable composition of the present invention for reducing facial folds. In an embodiment, the use of the present invention may be a cosmetic use, preferably a non-therapeutic use. The use of the present invention may be conducted by cosmetics, cosmetic professionals or health care professionals.

In a preferred embodiment, the use of an injectable composition of the present invention is for improvement of skin quality, treatment of fine lines, treatment of deep lines or volume restauration, or as super-volumizing filler for breast or buttock augmentation. The present invention also relates to a method of facial and body re-shaping and rejuvenation (preferably including the above specific uses), said method comprising administering the injectable composition according to the present invention.

When referring to a therapeutic use, the present invention relates to an injectable composition according to the present invention for use in a method for facial and body re-shaping and rejuvenation, preferably including filling of wrinkles, improving facial lines, breast reconstruction or augmentation, rejuvenation of the skin, buttocks augmentation, remodeling of cheekbones, soft-tissue augmentation, filling facial wrinkles, improving glabellar lines, improving nasolabial folds, improving marionette lines, improving buccal commissures, improving peri-lip wrinkles, improving crow's feet, improving subdermal support of the brows, malar and buccal fat pads, improving tear troughs, improving nose appearance, augmentation of lips, augmentation of cheeks, augmentation of peroral region, augmentation of infraorbital region, resolving facial asymmetries, improving jawlines, augmentation of chin, or a combination of two or more thereof.

In other words, the present invention also relates to a method for use in a method for facial and body re-shaping and rejuvenation, preferably including filling of wrinkles, improving facial lines, breast reconstruction or augmentation, rejuvenation of the skin, buttocks augmentation, remodeling of cheekbones, soft-tissue augmentation, filling facial wrinkles, improving glabellar lines, improving nasolabial folds, improving marionette lines, improving buccal commissures, improving peri-lip wrinkles, improving crow's feet, improving subdermal support of the brows, malar and buccal fat pads, improving tear troughs, improving nose appearance, augmentation of lips, augmentation of cheeks, augmentation of peroral region, augmentation of infraorbital region, resolving facial asymmetries, improving jawlines, augmentation of chin, or a combination of two or more thereof, wherein the a sufficient amount of the injectable composition according to the present invention is administered to a subject in need thereof.

As used herein, a subject (also: an individual) may be any animal, typically a mammal, preferably a domestic mammal or a human. Particularly preferably, an individual is a human. A treated human can also be designated as a patient, independent on his/her health state.

Administration may conducted by any means. In a preferred embodiment, administration is administration via a needle. In a preferred embodiment, administration is administration via a syringe, in particular intradermal or subdermal administration via a syringe. Administration may be manual administration, administration using a mechanical pump, or even automated administration. For instance, a Syringe One system may be used for administration.

The cross-linked material of the present invention as well as the injectable composition of the present invention may be used for any purpose. Optionally, the cross-linked material of the present invention as well as the injectable composition of the present invention may be used for cosmetic and/or therapeutic uses. The injectable composition may be a filler, in particular a soft tissue filler such as, e.g., soft-tissue filler, in particular a dermal filler or a connective tissue filler.

The present invention also refers to the use of an injectable composition according to the present invention as a filler such as a soft-tissue filler, in particular a dermal filler or a connective tissue filler. It may be used as a super-volumizer. In this context, it may be used as a hydrogel.

As used herein, the term “filler” may be understood in the broadest sense as any agent that can be used to fill a cavity or to serve as a soft tissue filler, preferably a soft tissue filler. A soft tissue filler may be understood in the broadest sense as a material designed to add volume to areas of soft tissue deficiency. A filler may be administered to any location and by any type of injection and may be suitable for uses in cosmetic/aesthetic applications as well as for therapeutic purposes. A filler may generally be any composition that adds, replaces or augments volume under the skin leading to, e.g., smoothened skin wrinkles, augmented lips, improved skin appearance, or treated scars. It is generally used in the dermis area, such as below the epidermis or above the hypodermis and as such may be injected subcutaneously, hypodermically or intradermally, or some combinations.

An injectable composition within the meaning of the present invention may be administered by means of (dispensed from) syringes under normal conditions under normal pressure. Moreover, the filler composition of the present invention is preferably (essentially) sterile. Preferably, the injectable composition is suitable for injection into a mammal, in particular a human.

Re-shaping may be performed for cosmetic purposes or may be performed after loss of tissue such as, e.g., caused by an accident or by a surgical intervention. For instance, a part of the face may be injured by an accident. On the other hand, cheekbones may be accentuated by filling the cheekbone region subcutaneously. A breast or part thereof may be surgically removed. On the other hand, breast reconstruction or augmentation may also have aesthetic reasons.

Preferably, the injectable composition of the present invention may be administered in an effective amount to an individual by injection, such as by subcutaneous or intradermal injection. In a preferred embodiment, in the context of this use, the injectable composition is a filler. In a more preferred embodiment, in the context of this use, the injectable composition is a filler, in particular a super-volumizer, and the use comprises the administration of the composition comprising the cross-linked material of the present invention in the tissue of interest, in particular subcutaneously or intradermally. For example, the injectable composition may be intradermally or subcutaneously injected using the serial puncture technique. An effective amount refers to the amount of the (injectable) soft tissue filler composition sufficient to effect beneficial or desired cosmetic (aesthetic) or therapeutic results.

In a particularly preferred embodiment, in the context of this use, the injectable composition is a filler which may be a super-volumizer, in particular a soft tissue filler, and the use comprises administration of the composition comprising the cross-linked material of the present invention subcutaneously or intradermally. For these uses, the cross-linked material according to the present invention is particularly beneficial because the cross-linked material rather stable in aqueous environments such as body fluids and enables invasion of cells due to its structure and characteristics.

A further aspect of the present invention relates to the cross-linked material or the injectable composition according to the present invention for use in a method for regenerating tissue of an individual in need thereof.

In other words, the present invention also relates to a method for regenerating tissue of an individual in need thereof, said method comprising administration of the cross-linked material or the injectable composition according to the present invention to the individual in need thereof. Regenerating tissue of an individual in need thereof may be performed for therapeutic and/or cosmetic purposes.

It will be understood that the definitions and preferred embodiments as laid out in the context of the cross-linked material, the hydrogel, the methods and the injectable composition above mutatis mutandis apply to the use of regenerating tissue of an individual.

The tissue to be regenerated may be any tissue. In one preferred embodiment, the tissue is a soft tissue. In a more preferred embodiment, the tissue is a soft tissue selected from the group consisting of dermal tissue (including tissue of the dermis and the subcutis) and connective tissue. Then, the method may be used for re-shaping and rejuvenation, including the uses as described above. In another preferred embodiment of the present invention, the tissue is an articulation (joint) tissue. Optionally, for this use, the cross-linked material may comprise one or more cell proliferation factors stimulating proliferation of the respective tissue.

In an alternative preferred embodiment, the tissue is bone tissue. Then, the cross-linked material of the present invention may be administered in a location where bone tissue is intended to grow such as e.g., in a gap of a bone fracture or for elongation of bones. Optionally, for this use, the cross-linked material may comprise one or more cell proliferation factors stimulating bone cell proliferation.

Depending on the specific use, the person skilled in the art will either use particulate cross-linked material according to the present invention or will use a block of the cross-linked material according to the present invention.

For the above therapeutic and cosmetic uses, the cross-linked material according to the present invention is particularly beneficial because the cross-linked material is rather stable in aqueous environments such as body fluids and enables invasion of cells due to its cross-linked structure and surface characteristics.

As indicated above, the cross-linked material of the present invention is obtained by the inventive material obtained when conjugating polysaccharide moieties. This conjugate as such also bears unexpectedly beneficial properties.

As used herein, the terms “approximately” and “about” may be understood as a scope including a deviation of up +/−10% of the respective number value. It will be understood that the specific values are also explicitly disclosed.

It will be further understood that the scope embraces the number values provided as commonly rounded values that embrace the whole rounding limits. For example, the scope of “1 mg” embraces the range of from 0.50 to 1.49 mg.

The number values of the present invention, however, also disclose the more detailed values of one or more orders of magnitude more in detail. Accordingly, for example, “1 mg” may also include the specific disclosure of “1.0 mg”.

The examples and figures illustrate embodiments of the present invention.

### BRIEF DESCRIPTION OF THE FIGURES

FIG. 1 shows an example of extrusion force measurements for a hyaluronic acid (HA) product cross-linked with 1.5 eq. of DMTMM. The measurements were performed using TSK 27 G ½″ needle at 12.6 mm/min. The figure shows an overlay of multiple measurements. It is visible that the measurements lead to comparable results and no significant inhomogeneities were found.

FIG. 2 shows degradation kinetics of cross-linked materials each based on hyaluronic acid (HA) which was cross-linked by different contents of DMTMM. The degradation was performed with contacting the respective cross-linked material with 150 units of hyaluronidase and incubation at 37° C. The upper graphs show HA obtained from a first supplier. The lower graphs show HA obtained from another supplier. The triangle pointing downwards indicate non-cross-linked HA. The squares indicate HA cross-linked with 0.5 equivalents (eq.) of DMTMM. The circles indicate HA cross-linked with 1.0 eq. of DMTMM. The triangle pointing upwards indicate HA cross-linked with 1.5 eq. of DMTMM.

### EXAMPLES

### Example 1

### Methods

Preparation of cross-linked hyaluronic acid material according to the invention 1.5 g hyaluronic acid sodium salt (HA) (dry weight) with intrinsic viscosity of 2.85 m³/kg was solubilized in 45 mL phosphate buffered saline (PBS) buffer of pH 7.4. The obtained mixture was pre-swelled for approximately 15 hours. This was noted to be an optional step that could also be omitted.

Different contents (for instance approximately 0, 0.2, 0.5, 1.0, 1.5 or 2.0 molar equivalents (eq.) related to the carboxyl groups of HA) of the triazin-based activating agent 4-(4,6-dimethoxy-1,3,5-triazin-2-yl)-4-methylmorpholinium chloride (DMTMM) were dissolved in each 5 mL of PBS buffer of pH 7.4. 1.03 g of DMTMM was used to achieve approximately 1 eq. referred to carboxyl groups of HA.

The 45 mL of PBS buffer containing (HA) and the 5 mL of PBS buffer containing DMTMM were mixed with each other. This achieves a HA concentration of 30 mg/mL. Mixing was conducted until a visually homogeneous composition was achieved. This was typically achieved after approximately 10 min.

In the experiments depicted herein, the sample was maintained at 40° C. for 3 hours. However, it was noted that reaction was also well workable at other temperatures such as at ambient temperature of approximately 20° C.-22° C. It was further observed that also shorter reaction times also resulted in feasible cross-linking material.

Subsequently, the cross-linked material (present as a hydrogel) was homogenized once more for 30 min and passed through a sieve (screening). This was noted to be an optional step that could also be omitted.

Subsequently, the cross-linked material (present as a hydrogel) was placed in dialysis membranes (molecular weight cut-off (MWCO) 12-14 kDa) and was purified for approximately 2 days to remove the side products of the DMTMM. The swelling ratio was controlled by the weight of the membranes and the dialysis stopped once the product showed the desired cross-linked material concentration. This was noted to be an optional step that could also be omitted.

Optionally, a local anesthetic such as lidocaine is added to the cross-linked material (present as a hydrogel). Optionally, the cross-linked material (present as a hydrogel) is subjected to sterilization and optionally filled in syringes.

### Determination of Properties

Rheology (including intrinsic viscosity) was determined using a 302 Modular Compact Rheometer (Anton Paar GmbH, Graz, Austria). Briefly, plate/plate in oscillation mode (PP20) with a 1.0 mm gap size at 25° C. was used; Tau (stress) was set to 10 Pa, and the oscillation frequency ranged from 10 to 0.1 Hz. Reported G′ values correspond to 1 Hz.

Extrusion force was measured using a TA.XT. (Stable Micro Systems) Plus texture analyzer. Briefly, syringes were extruded at constant speed (12.6 mm/min), and the forces (in N) were measured over time. A-½ inch 27 G TSK (TSK Laboratory, Japan) needle,

Enzymatic Degradation: The material degradation was evaluated though Rheometer Anton Paar MCR 302, plate CP50-1 at 36° C. in the oscillation modus. The measurement points were recorded each minute for 1 hour. The content of one syringe (1 mL) was deposited on the measuring plate and 150 μL of hyaluronidase solution with 150 U (activity units) was mixed in the gel for 30 seconds. The G′ values at 1 Hz were converted in degradation % to report the values.

Molecular weight values and distributions were determined by Gel Permeation Chromatography (GPC) calibrated to pullulan standards, using samples with a concentration of 5 mg/mL in PBS buffer (pH 7.4).

### Results

It was found that cross-linked material with beneficial properties is obtainable at various content ratios.

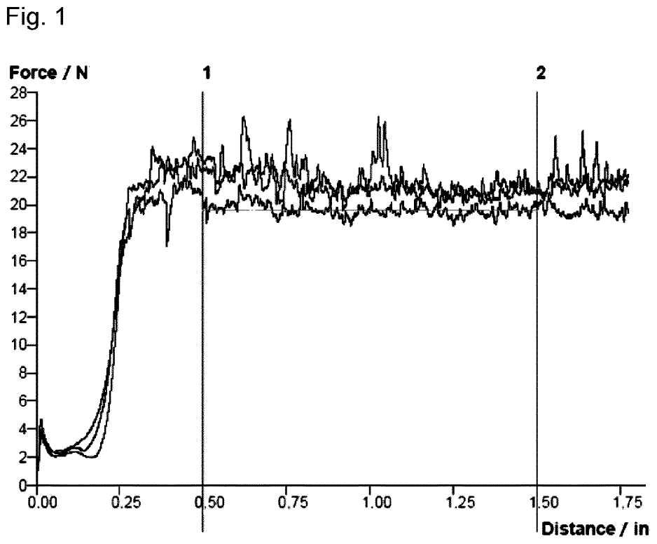

A summary of the results using small scale experiments is shown in the Table 1 below. The rheological parameters are consistent to other polysaccharide-based hydrogels (e.g., HA fillers) and there is a drop in the in the G′ values of circa 50% after the sterilization. Representative extrusion curves are shown in FIG. 1 for a gel crosslinked with 1.5 eq. of activating agent (DMTMM). Without being bound to this theory, it appears that the cross-linked material displays a shear thinning effect due to the low extrusion forces.

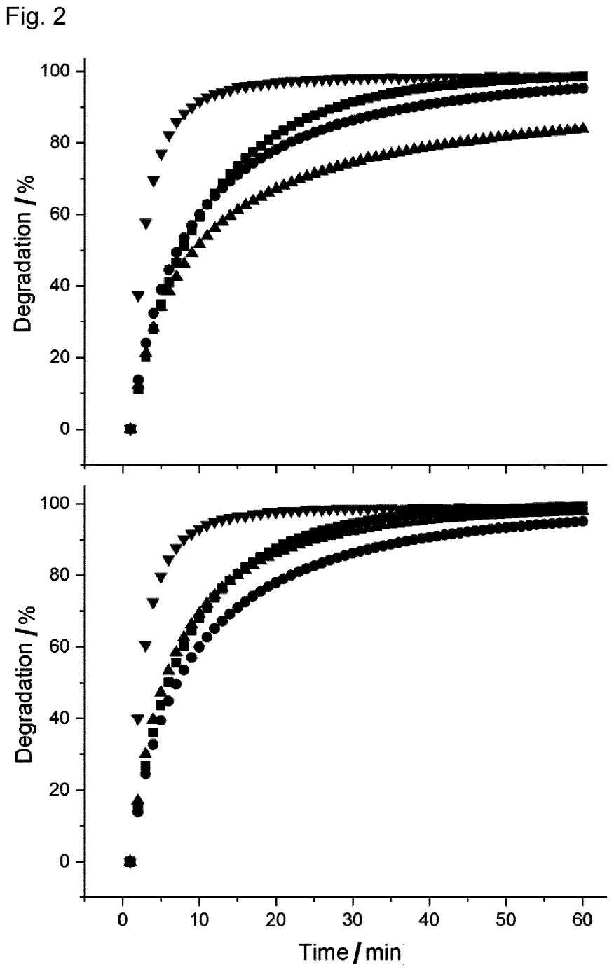

The enzymatic degradation of the different products was further investigated. The results are depicted in FIG. 2. Herein, it is visible that broad content ratios of polysaccharide moieties and activating agents can be used.

It was found that the cross-linked material using less activating agent (DMTMM) equivalents show lower degree of cross-linking. The cross-linked material having a lower degree of cross-linking could be essentially completely degraded by the enzyme after one hour, while the cross-linked material having a higher degree of cross-linking were more stable. This shows that the method of the present invention is well suitable to purposefully adjust the cross-linking degree to desired purposes.

### Example 2

### Preparation of a Cross-Linked Chondroitin Sulphate Material According to the Invention

In the same manner as for Example 1, 2.0 g of chondroitin sulphate (CS) with a weight average molecular weight of 100 kDa was solubilized in PBS buffer (pH 7.4). Different contents (approximately 0.0, 0.5, 1.0 or 1.5 molar equivalents (eq.) related to the carboxyl groups of CS) of DMTMM were dissolved in PBS buffer of pH 7.4 and mixed with the PBS buffer containing CS, until a visually homogeneous composition was achieved, and the mixture was allowed to react at 23° C. for 17 h. Subsequently, the crosslinked hydrogel was purified by dialysis using PBS buffer. 1.15 g of DMTMM was used to achieve approximately 1 eq. referred to carboxyl groups of CS. The rheological properties were determined as described above for Example 1, and are shown in the Table 2.

TABLE 1
Summary of the results using two different batches of hyaluronic acid (HA) (each 30 mg/mL) and different molecular
equivalents (eq.) of activating agent DMTMM. The results show rheology values of sterile and non-sterile product

| | Used content | | | | | | | | | | | |
|---|---|---|---|---|---|---|---|---|---|---|---|---|
| | ranges | | | | | | | | | Determined HA | | |
| | | DMTMM | non-sterile | | | | sterile | | | Extru- | HA | |
| Experiment number | Used | (cross- | | | tan | G′ | | | tan | sion | concen- | Free |
| Experiment number | HA | linking) | Iη*I | G′ | (δ) | Drop | Iη*I | G′ | (δ) | force | tration | HA |
| (HA lot) | [g] | [eq.] | [1.0 Hz] | | | [%] | [1.0 Hz] | | | [N] | [mg/g] | [%] |
| Pure HA-A (4368C) | 1.444 | (−) | 77.8 | 441 | 0.479 | 55.1 | 42.7 | 198 | 0.917 | 6.2 | | |
| Pure HA-B (20011C) | 1.35 | (−) | 95.1 | 547.5 | 0.437 | 41.5 | 61.1 | 320 | 0.667 | 5.5 | | |
| HAHA221101-A | 1.627 | 0.5 (+) | 131.5 | 822.4 | 0.100 | 65.3 | 48.8 | 285 | 0.391 | 15.5 | 24.4 | 47.7 |
| (20011C) | | | | | | | | | | | | |
| HAHA221101-B | 1.623 | 1 (+) | 135.6 | 849.8 | 0.072 | 44.6 | 75.9 | 470 | 0.166 | 17.6 | 24.4 | 12.5 |
| (20011C) | | | | | | | | | | | | |
| HAHA221101-C | 1.621 | 1.5 (+) | 149.4 | 937.1 | 0.064 | 55.1 | 67.5 | 421.3 | 0.111 | 20.9 | 21.7 | 13.1 |
| (20011C) | | | | | | | | | | | | |
| HAHA221101-D | 1.732 | 0.5 (+) | 120.1 | 748.7 | 0.126 | 61.93 | 67.8 | 285 | 0.276 | 14.56 | 25.0 | 22.9 |
| (4368C) | | | | | | | | | | | | |
| HAHA221101-E | 1.732 | 1 (+) | 130.31 | 816.3 | 0.077 | 49.78 | 46.29 | 410.00 | 0.203 | — | 25.7 | 19.4 |
| (4368C) | | | | | | | | | | | | |
| HAHA221101-F | 1.728 | 1.5 (+) | 140.37 | 880.3 | 0.061 | 47.49 | 74.23 | 462.30 | 0.133 | — | 19.5 | 9.4 |
| (4368C) | | | | | | | | | | | | |

TABLE 2
Summary of the rheological properties of chondroitin
sulphate before cross-linking and after cross-linking
using different molecular equivalents (eq.) of the
activating agend DMTMM (non-sterile product).

| | Experiment number | | | | |
|---|---|---|---|---|---|
| | Used content ranges | | non-sterile | | |
| | Experiment number | | | | |
| | | DMTMM | | | |
| | | (cross- | | | |
| | Used CS | linking) | \|η*\| | G′ | tan (δ) |
| | [g] | [eq.] | [1.0 Hz] | | |
| Pure chondroitin | 2.0 | (—) | 0.05 | 0.04 | 7.06 |
| sulphate (before | | | | | |
| cross-linking) | | | | | |
| A | 2.0 | 0.5 | 5.5 | 33 | 0.35 |
| B | 2.0 | 1.0 | 35.1 | 220 | 0.09 |
| C | 2.0 | 1.5 | 49.6 | 311 | 0.05 |

1-15. (canceled)

16. A method for preparing a cross-linked material comprising:
(i) contacting the following components with each other:
(A) polysaccharide moieties comprising carboxylic acid residues or salts thereof and hydroxy residues;
(B) one or more triazine-based activating agents that effect reaction of carboxylic acid residues with hydroxy residues to form ester bonds; and
(C) one or more solvents,
(ii) allowing reaction of at least some of the carboxylic acid residues of the polysaccharide moieties with at least some of the hydroxy residues of the polysaccharide moieties to form ester bonds cross-linking the polysaccharide moieties covalently with each other, and
(iii) optionally, purifying the cross-linked material obtained from step (ii),
wherein there are more ester bonds cross-linking the polysaccharide moieties covalently with each other than amide bonds cross-linking the polysaccharide moieties covalently with each other, and
wherein step (ii) is conducted in the absence of carbodiimide activating agents, succinimidyl-based activating agents, glycidyl-based activating agents, paranitrophenol esters, and 2-chloro-methylpyridinium iodide.

17. The method of claim 16, wherein the polysaccharide moieties comprise a molar ratio of hydroxy residues:primary amine groups of at least 25:1.

18. The method of claim 16, wherein the polysaccharide moieties comprise hyaluronic acid, glycerol-grafted hyaluronic acid, heparosan, chondroitin sulfate, or carboxymethyl cellulose.

19. The method of claim 16, wherein the polysaccharide moieties comprise one or more hyaluronic acid moieties.

20. The method of claim 16, wherein the polysaccharide moieties have a weight average molecular weight of at least 50 kDa.

21. The method of claim 16, wherein the one or more triazine-based activating agents are selected from the group consisting of 4-(4,6-dimethoxy-1,3,5-triazin-2-yl)-4-methylmorpholinium or a salt thereof, 2-chloro-4,6,-dimethoxy-1,3,5-triazine or a salt thereof, and combinations thereof.

22. The method of claim 21, wherein the one or more triazine-based activating agents are 4-(4,6-dimethoxy-1,3,5-triazin-2-yl)-4-methylmorpholinium or a salt thereof.

23. The method of claim 16, wherein the one or more solvents are an aqueous solution.

24. The method of claim 16, wherein the one or more solvents are an aqueous buffer having a pH in the range of 5 to 9.

25. The method of claim 16, wherein the cross-linked material is prepared without at least one of:
interconnecting linker moieties inserted between the carboxylic acid residues or salts thereof and the hydroxy residues of the polysaccharide moieties;
a carbodiimide-based activating agent;
reactive groups selected from the group consisting of glycidyl ethers, maleimides, acid anhydrides, alkoxides, and combinations of two or more thereof; or
polysaccharide moieties having primary amine groups, thiol groups, imide groups, imine groups, or epoxy groups.

26. The method of claim 16, wherein:
optional step (iii) is conducted and the cross-linked material is purified by filtration, washing, and/or dialysis;
steps (i) and (ii) are conducted in a single batch;
steps (i) and (ii) and optional step (iii) are conducted at a temperature in the range of from 5 to 90° C.;
step (ii) is conducted for at least 10 min; or
any combination thereof is conducted.

27. The method of claim 16, wherein:
the polysaccharide moieties have a weight average molecular weight of at least 50 kDa, and
the one or more solvents are an aqueous solvent.

28. A cross-linked material obtained from the method of claim 16.

29. A cross-linked material comprising hyaluronic acid moieties having a weight average molecular weight of at least 50 kDa,
wherein the hyaluronic acid moieties are covalently cross-linked with each other via ester bonds without an interconnecting linker structure, and
wherein the cross-linked material is free of diimide groups, epoxy groups, and xenobiotic linker moieties.

30. The cross-linked material of claim 29, wherein the cross-linked material further comprises:
4-(4,6-dimethoxy-1,3,5-triazin-2-yl)-4-methylmorpholinium or a salt thereof;
N-methylmorpholinium or a salt thereof;
4,6-dimethoxy-1,3,5-triazin-2-ol or a tautomer or a salt thereof; or
combinations thereof.

31. A hydrogel comprising:
(A) the cross-linked material of claim 29;
(B) an aqueous solution; and
(C) optionally, one or more further pharmaceutically or cosmetically acceptable agents.

32. An injectable composition comprising the cross-linked material of claim 29, wherein the cross-linked material is a super-volumizer.

33. The injectable composition of claim 32, wherein the cross-linked material is part of a hydrogel further comprising an aqueous solution and optionally, one or more further pharmaceutically or cosmetically acceptable agents.

34. A method of facial or body re-shaping or rejuvenation comprising:
administering to a subject the injectable composition of claim 33.

35. The method of claim 34, wherein the facial or body re-shaping or rejuvenation is selected from the group consisting of filling of wrinkles, improving facial lines, breast reconstruction or augmentation, rejuvenation of skin, buttocks augmentation, remodeling of cheekbones, soft-tissue augmentation, filling facial wrinkles, improving glabellar lines, improving nasolabial folds, improving marionette lines, improving buccal commissures, improving peri-lip wrinkles, improving crow's feet, improving subdermal support of brows, malar, and buccal fat pads, improving tear troughs, improving nose appearance, augmentation of lips, augmentation of cheeks, augmentation of peroral region, augmentation of infraorbital region, resolving facial asymmetries, improving jawlines, augmentation of chin, and a combination of two or more thereof.
